(19) United States
(12) Patent Application Publication
(10) Pub. No.: US 2012/0128815 A1
(43) Pub. Date: May 24, 2012

(54) PERFORMANCE ENHANCING SPORTS BEVERAGE AND METHODS OF USE

(75) Inventors: Sylvia P. Poulos, Decatur, GA (US); Julio Boza, Marietta, GA (US); Robert O. Scott, Douglasville, GA (US)

(73) Assignee: The Coca-Cola Company, Atlanta, GA (US)

(21) Appl. No.: 13/299,493

(22) Filed: Nov. 18, 2011

Related U.S. Application Data
(60) Provisional application No. 61/415,016, filed on Nov. 18, 2010.

(51) Int. Cl. A23L 2/52; A23L 1/304

(52) U.S. Cl. 426/2; 426/74; 426/590; 426/548; 426/72

(57) ABSTRACT

A performance enhancing sports drink is described. The drink, which is useful before, during, and after exercise, is specially developed to reduce lactate production, reduce perceived exhaustion, reduce muscle soreness (both actual muscle damage and perceived soreness), and reduce net fluid loss observed with exercise.

This application claims the benefit of U.S. Prov. Appl. No. 61/415,016, filed Nov. 18, 2010, the entire disclosure of which is incorporated herein by reference.

### FIELD OF INVENTION

A performance enhancing sports drink is described. The drink, which is useful before, during, and after exercise, is specially developed to reduce lactate production, reduce perceived exhaustion, reduce muscle soreness (both actual muscle damage and perceived soreness), and reduce net fluid loss observed with exercise.

### BACKGROUND OF INVENTION

Most products aimed at enhancing sports performance have focused on optimizing carbohydrate availability as a way to reduced glycogen depletion. However it is known that one physiological response to endurance training is the enhanced oxidation of fat at rest and during exercise. (Scharhag-Rosenberger et al., 2010; Jeukendrup, 2003). More studies have been conducted to understand the role of increased lipid stores found in the skeletal muscle of athletes as compared to sedentary individuals and researchers have suggested this is a labile energy store used during exercise. Several studies suggest that during moderate exercise, approximately half of the total fat oxidized appears to be from circulation in endurance-trained men and the amount of fat oxidized is significant. (Romijn et al., 1993; Sidossis et al., 1998; Coyle et al., 2001; van Loon et al., 2001). These studies imply that increasing the amount of fatty acids available for oxidation during exercise may enhance endurance exercise performance.

Medium chain triacylgylcerols (“MCTs”) are lipids whose fatty acid chains are 6-12 carbons in length. These lipids are adsorbed quickly and have a metabolic profile that mimics carbohydrates. Although some studies have suggested that MCTs may improve physical performance, athletes consuming more than 30 grams of these lipids have increased risk for gastrointestinal disturbances. In addition, these lipids are relatively expensive and may be cost prohibitive for formulations.

Long chain triacylglycerols (“LCTs”) are lipids that contain more than 12 carbon atoms in their aliphatic tail. While lipid infusion using LCTs during exercise has been shown to delay glycogen depletion, several studies have shown that oral intake of LCTs before and during exercise does not improve physical performance in well-trained athletes. (Whitley et al., 1998; Jeukendrup et al., 2004; Horowitz et al., 2000). In addition, product formulations with substantial levels of LCTs may cause undesirable beverage attributes. Therefore, supplementation of sports beverages with LCTs for enhanced sports performance is not recommended.

Short chain fatty acids are lipids with aliphatic tails of less than six carbons long and include acetic acid, propionic acid, isobutyric acid, butyric acid, isovaleric acid, valeric acid, and caproic acid. Short chain fatty acids, including acetic acid, are found in the diet and are also produced in the gastrointestinal tract through bacteria fermentation of ingested foods.

Acetic acid, also known as ethanoic acid is commonly consumed in vinegar. It is a weak organic acid and a short chain fatty acid. While many historic uses have little or no scientific efficacy, vinegar has traditional and historic use purportedly because it reduces inflammation, reduces blood pressure, increases mineral absorption, and helps recovery from fatigue.

In the food industry, acetic acid is used under the food additive code E260 as an acidity regulator. Alternatively, the synthetic triglyceride triacetin (glycerin triacetate) is also a common food additive containing an acetate source and is commonly used as a flavor stabilizer that does not provide acidity.

In contrast to LCTs, short chain fatty acids are absorbed directly through the portal vein after digestion. Intracellularly, acetate may be enzymatically converted to acetyl coenzyme A, which may then enter the Krebs cycle to generate adenosine triphosphate (ATP), the main energy molecule of a cell. Thus, the use of short chain fatty acids may be an alternative for providing energy to muscles during exercise. Suggestive evidence of efficacy for increased physical performance is found in several animal studies. (Fushimi et al., 2001 and 2002; Roberts et al., 2005).

Thus, it would be beneficial to develop a sports drink that contains both acetate and carbohydrate, as opposed to just carbohydrate, to improve athletic performance.

### SUMMARY OF INVENTION

A method of enhancing athletic performance comprising consuming a sports beverage before, during, or after exercise is provided. In another aspect, a sports beverage and a sports food are provided.

In some embodiments, the invention relates to a method of enhancing athletic performance comprising consuming a sports beverage before, during, or after endurance exercise, wherein the sports beverage comprises an aqueous solution of a carbohydrate source in an amount ranging from about 1.0 wt % to about 10.0 wt % of the sports beverage; an acetate source in an amount ranging from about 5.0 mM/L to about 40.0 mM/L; and one or more electrolytes in an amount ranging from about 30 mM/L to about 180 mM/L. In additional embodiments, said electrolytes are present in an amount including but not limited to about 40 mM/L, about 50 mM/L, about 60 mM/L, about 70 mM/L, about 80 mM/L, about 90 mM/L, about 100 mM/L, about 110 mM/L, about 120 mM/L, about 130 mM/L, about 140 mM/L, about 150 mM/L, about 160 mM/L and about 170 mM/L. In further embodiments, said carbohydrate source is selected from the group consisting of high fructose corn syrup, sucrose, fructose, maltodextrin, glucose, fruit juice, and combinations thereof. In still further embodiments, said acetate source is selected from the group consisting of acetic acid, acetate, vinegar, acetate anhydride, calcium acetate, potassium acetate, sodium acetate, triacetin (also known as glycerin triacetate; 1,3-diacetyloxypropan-2-yl acetate or 1,2,3-triacetoxypropane), salts thereof, and combinations thereof. In additional embodiments, said electrolytes are selected from the group consisting of sodium, potassium, chloride, calcium, magnesium, bicarbonate, phosphate, sulfate, and combinations thereof. In some embodiments, said carbohydrate source is present in an amount ranging from about 2.0 wt % to about 8.0 wt %, including but in no way limited to about 3 wt %, about 4 wt %, about 5 wt %, about 6 wt %, and about 7 wt %, of said sports beverage. In further embodiments, said carbohydrate source is high fructose corn syrup. In still further embodiments, said acetate source is present in an amount ranging from about 3.0 mM/L to about 28.0 mM/L, including but in no way limited to about 4 mM/L, about 5 mM/L, about 6 mM/L, about 7 mM/L, about 8 mM/L, about 9 mM/L, about 10 mM/L, about 12 mM/L and about 15 mM/L. In additional embodiments, said acetate source is present in an amount ranging from about 4.0 mM/L to about 5.0 mM/L. In further embodiments, said sports beverage further comprises one or more coloring additives, one or more flavor additives, one or more artificial or non-caloric sweeteners, one or more vitamins, one or more nutritional supplements, and combinations thereof. In still further embodiments, said enhancing athletic performance is characterized by reduced perceived exhaustion, reduced lactic acid production, reduced muscle soreness, reduced muscle damage, reduced net fluid loss, or combinations thereof.

In some embodiments, the invention relates to a sports beverage comprising an aqueous solution of a carbohydrate source in an amount ranging from about 1.0 wt % to about 10.0 wt % of the sports beverage; an acetate source in an amount ranging from about 5.0 mM/L to about 40.0 mM/L; and one or more electrolytes in amount ranging from about 30 mM/L to about 180 mM/L. In additional embodiments, said electrolytes are present in an amount including but not limited to about 40 mM/L, about 50 mM/L, about 60 mM/L, about 70 mM/L, about 80 mM/L, about 90 mM/L, about 100 mM/L, about 110 mM/L, about 120 mM/L, about 130 mM/L, about 140 mM/L, about 150 mM/L, about 160 mM/L and about 170 mM/L. In further embodiments, said carbohydrate source is selected from the group consisting of high fructose corn syrup, sucrose, fructose, maltodextrin, glucose, fruit juice, and combinations thereof. In still further embodiments, said acetate source is selected from the group consisting of acetic acid, acetate, vinegar, acetate anhydride, calcium acetate, potassium acetate, sodium acetate, triacetin, and combinations thereof. In additional embodiments, said electrolytes are selected from the group consisting of sodium, potassium, chloride, calcium, magnesium, bicarbonate, phosphate, sulfate, and combinations thereof. In some embodiments, said carbohydrate source is present in an amount ranging from about 2.0 wt % to about 8.0 wt %, including but in no way limited to about 3 wt %, about 4 wt %, about 5 wt %, about 6 wt %, and about 7 wt %, of said sports beverage, of the sports beverage. In further embodiments, said carbohydrate source is present in an amount ranging from about 4.0 wt % to about 8.0 wt % of the sports beverage. In still further embodiments, said carbohydrate source is high fructose corn syrup. In additional embodiments, said acetate source is present in an amount ranging from about 3.0 mM/L to about 6.0 mM/L, including but in no way limited to about 4 mM/L and about 5 mM/L. In some embodiments, said acetate source is present in an amount ranging from about 4.0 mM/L to about 5.0 mM/L. In further embodiments, the sports beverage further comprises one or more coloring additives, one or more flavor additives, one or more artificial or non-caloric sweeteners, one or more vitamins, one or more nutritional supplements, and combinations thereof. In still further embodiments, the invention relates to a method for enhancing performance comprising orally administering the sports beverage of claim 11 to an endurance athlete before, during, or after said endurance athlete engages in endurance exercise, wherein the enhanced performance is characterized by reduced perceived physical exhaustion, reduced muscle soreness, reduced muscle damage, reduced net fluid loss, reduced production of lactic acid, or combinations thereof.

Additional aspects will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

FIG. 1 shows the effects of the ingestion of control, acetic acid, or triacetin beverages on plasma glucose at baseline (BL) and during Segments 1-4 (S1-S4) of the endurance cycle test. Values are expressed as mean±SEM (n=11).

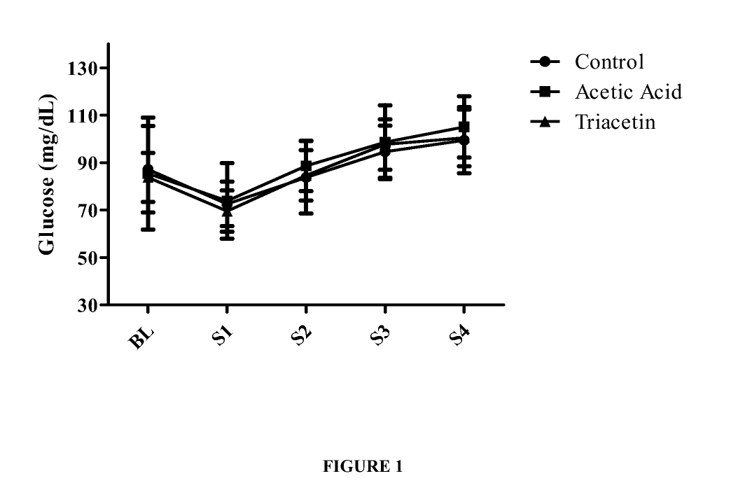

FIG. 2 shows the effects of the ingestion of control, acetic acid, or triacetin beverages on plasma lactate at baseline (BL) and during Segments 1-4 (S1-S4) of the endurance cycle test. Values are expressed as mean±SEM (n=11).

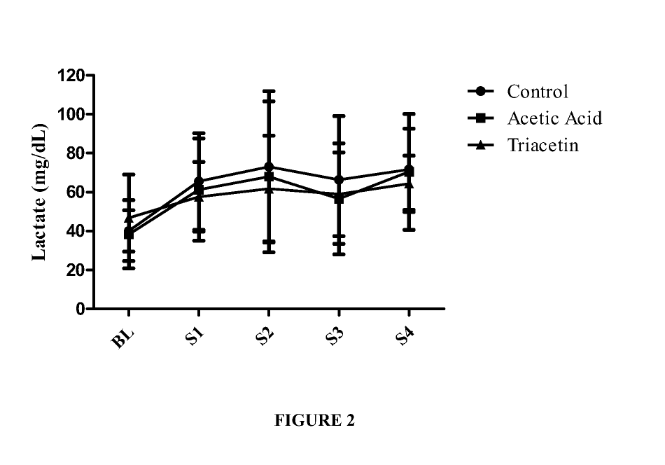

FIG. 3 shows the effects of the ingestion of control beverage (water), sports beverage, or sports beverage+acetic acid on plasma lactate during a staged exercise performance test consisting of a pre-exercise period prior to any physical activity; a 30-min exercise preload period conducted at pooled intensities of 55 and 65% VO_{2MAX}; a 4-min active recovery period and a 12-min time trial. Values are expressed as mean±SEM (n=50 for all treatments and time periods, with the exception of pre-load period with sports beverage, n=49). ** Sports beverage and sports beverage+acetate significantly (p<0.01) different from control.

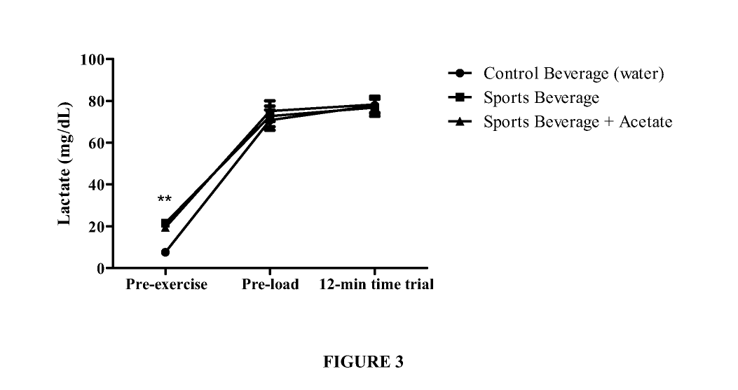

FIG. 4 shows the effects of the ingestion of control, acetic acid, or triacetin beverages on mean minute ventilation at baseline (BL) and during Segments 1-4 (S1-S4) of the endurance cycle test. Values are expressed as mean±SEM (n=11). *Triacetin differed significantly (p=0.032) from control.

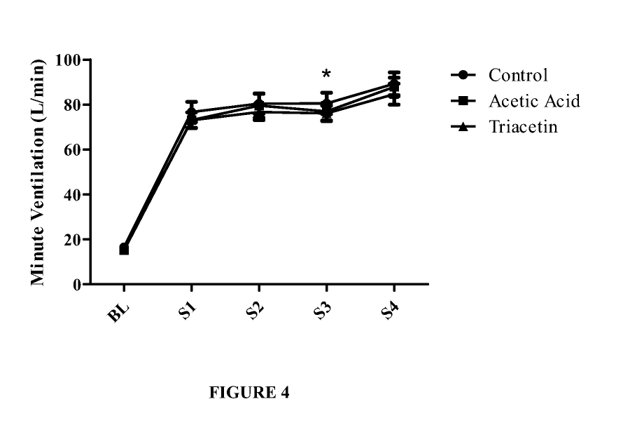

FIG. 5 shows the effects of the ingestion of control beverage (water), sports beverage, or sports beverage+acetic acid on mean minute ventilation during an exercise performance test consisting of a pre-exercise period prior to any physical activity; a 30-min exercise preload period conducted at pooled intensities of 55% and 65% VO_{2MAX}; a 4-min active recovery period and a 12-min time trial. Values for preload average represent the average of time points take at the mid-point and end of the preload. Values for the 12-min time trial represent the average values collected during that period. Values are expressed as mean±SEM (n=50). *Sports beverage+acetate significantly (p<0.05) different from control; ** Sports beverage and sports beverage+acetate significantly (p<0.01) different from control

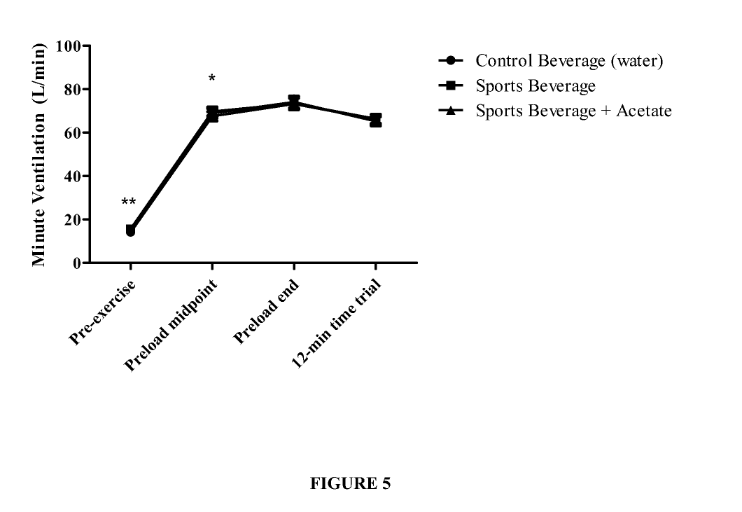

### DETAILED DESCRIPTION

Disclosed herein are a method of enhancing athletic performance with a sports beverage composition and a sports beverage composition. It has been found that blood glucose levels and blood lactate levels may be reduced when athletes consume beverages containing acetate. Surprisingly, the acetate containing beverages also tend to reduce oxygen uptake measured as liters of air inspired per minute. These findings suggest that acetate can alter substrate metabolism in healthy adults and may enhance the athletic performance of athletes during endurance exercise by providing a rapidly metabolized fuel, reducing net fluid loss, reducing the appearance of lactate in the blood, and reducing minute ventilation.

The term “beverage” as used herein means any drinkable liquid or semi-liquid, including for example water, flavored water, soft drinks, fruit drinks, tea-based drinks, juice-based drinks, gel drinks, carbonated or non-carbonated drinks, and alcoholic or non-alcoholic drinks. In some embodiments, a beverage powder may first be mixed with any drinkable liquid or semi-liquid to obtain a beverage.

Method of Enhancing Athletic Performance with a Sports Beverage

In one aspect, a method of enhancing athletic performance comprising consuming a sports beverage is provided. As used herein, “enhanced athletic performance” refers to an improvement in athletic performance associated with consuming embodiments of the sports beverages provided herein, as compared to athletic performance without consuming the sports beverage or water. The sports beverage may be consumed before, during, or after athletic performance. As used herein, “athletic performance” refers to both endurance exercise and non-endurance exercise. Endurance exercise includes aerobic activities over prolonged periods of time (e.g., greater than about 30 minutes) while non-endurance exercise includes aerobic activities over a shorter period of time (e.g., less than about 30 minutes).

As used herein, “control beverage” refers to a beverage that contains both a carbohydrate source and one or more electrolytes including sodium, but does not contain an acetate source.

In one embodiment, enhanced performance may be characterized by a reduced perception of physical exhaustion (perceived exertion), subjectively characterized by the Borg perceived exertion scale. In some embodiments, consuming the sports beverage results in perceived exertion that is reduced by about 1% to about 5% on the Borg scale, relative to perceived exertion when consuming a control beverage or water.

In another embodiment, enhanced performance may be characterized by a reduced muscle soreness, as subjectively felt. In some embodiments, consuming the sports beverage results in a reduced feeling of muscle soreness, relative to the feeling of muscle soreness when consuming a control beverage or water.

In still another embodiment, enhanced performance may be characterized by a reduced net fluid loss. In some embodiments, consuming the sports beverage results in a volume of fluid loss that is reduced by at least about 5%, at least about 10%, or at least about 20%, relative to the volume of fluid loss when consuming a control beverage or water

In yet another embodiment, enhanced performance may be characterized by a reduced production of lactic acid. In some embodiments, consuming the sports beverage results in a reduction of the production of lactic acid by at least about 5%, at least about 10%, or at least about 15%, relative to the production of lactic acid when consuming a control beverage or water.

In other embodiments, enhanced performance may be characterized by performing exercise for a longer period of time, performing exercise at a higher intensity, performing exercise with a higher power output, performing a specific exercise task in a reduced amount of time, increased exercise performance in a given amount of time, and the like.

In other embodiments, enhanced performance may be characterized by one or more of the foregoing.

Sports Beverage

The sports beverages provided herein generally comprise an aqueous solution of at least one carbohydrate source, at least one acetate source, and one or more electrolytes, including sodium.

In some embodiments, the aqueous solution may comprise tap water. In other embodiments, the aqueous solution may comprise deionized water. In still other embodiments, the aqueous solution may comprise spring water.

In some embodiments, the carbohydrate source includes, but is not limited to, high fructose corn syrup, sucrose, fructose, maltodextrin, glucose, or combinations thereof. Other carbohydrate sources include mono-saccharides, dextrose, maltose, dextrin, xylose, ribose, mannose, galactose, lactose, invert sugar, tagatose, sugar alcohols such as glycerol, sorbitol, xylitol, mannitol, galactitol, maltitol, lactitol, erythritol, hydrogenated starch hydrolase, polyglycitol, stevia, fruit juice, or combinations thereof. In particular embodiments, the carbohydrate source is present in the sports beverage in an amount in the range of about 1.0 wt % to about 10.0 wt %, about 1.0 wt % to about 8.0 wt %, about 2.0 wt % to about 8.0 wt %, or about 4.0 wt % to about 8.0 wt %. The carbohydrate desirably functions as both a sweetener and a source of energy.

In some embodiments, the acetate source includes, but is not limited to, vinegar, acetic acid, acetate anhydride, calcium acetate, potassium acetate, sodium acetate, triacetin, or combinations thereof. In still other embodiments, the acetate source may be a short chain fatty acid, including but not limited to propionic acid, isobutyric acid, butyric acid, isovaleric acid, valeric acid, caproic acid, or combinations thereof. In particular embodiments, the acetate source is present in the sports beverage in an amount in the range of about 3.0 mM/L to about 28.0 mM/L, about 3.0 mM/L to about 20.0 mM/L, about 3.0 mM/L to about 10.0 mM/L, or about 4.0 mM/L to about 5.0 mM/L.

In some embodiments, the electrolyte includes, but is not limited to, sodium, potassium, chloride, calcium, magnesium, bicarbonate, phosphate, sulfate, or combinations thereof. In particular embodiments, sodium is present in the sports beverage in an amount in the range of about 30 mM/L to about 180 mM/L, from about 60 mM/L to about 150 mM/L, or from about 80 mM/L to about 150 mM/L.

The sodium electrolyte may be in the form of, but is not limited to, sodium chloride, sodium acetate, sodium citrate, sodium phosphate, sodium bicarbonate, sodium bromide, sodium citrate, sodium lactate, sodium sulfate, sodium tartrate, sodium benzoate, sodium selenite, or combinations thereof.

The potassium electrolyte may be in the form of, but is not limited to, potassium chloride, potassium acetate, potassium bicarbonate, potassium bromide, potassium citrate, potassium-D-gluconate, potassium phosphate, potassium tartrate, potassium sorbate, potassium iodide, or combinations thereof.

The magnesium electrolyte may be in the form of, but is not limited to, magnesium chloride, magnesium oxide, magnesium sulfate, magnesium aspartate, magnesium silicate, or combinations thereof.

The chloride electrolyte may be in the form of, but is not limited to, sodium chloride, potassium chloride, magnesium chloride, calcium chloride, or combinations thereof.

The calcium electrolyte may be in the form of, but is not limited to, calcium chloride, calcium oxide, calcium sulfate, calcium phosphate, calcium lactate, calcium gluconate, or combinations thereof.

The bicarbonate electrolyte may be in the form of, but is not limited to, sodium bicarbonate, potassium bicarbonate, or combinations thereof.

The phosphate electrolyte may be in the form of, but is not limited to, sodium phosphate, potassium phosphate, calcium phosphate, or combinations thereof.

The sulfate electrolyte may be in the form of sodium sulfate, potassium sulfate, magnesium sulfate, calcium sulfate, or combinations thereof.

In still other embodiments, the sports beverage may further comprise one or more coloring additives, one or more flavoring additives, one or more artificial or non-caloric sweeteners, one or more vitamins, one or more nutritional supplements, or combinations thereof.

Non-limiting examples of coloring additives include, but are not limited to, natural food dyes or extracts, artificial colorings, dye pigments, or combinations thereof.

Non-limiting examples of flavor additives include, but are not limited to, fruit juices or fruit juice concentrates, and aldehydes and esters (e.g., cinnamyl acetate, cinnamaldehyde, acetaldehyde, benzaldehyde, citral, decanal, ethyl vanillin, piperonal, vanillin, and 2-dodecenal), or combinations thereof.

Non-limiting examples of artificial or non-caloric sweeteners include, but are not limited to, aspartame, saccharin, sucralose, acesulfame potassium, or stevia.

Non-limiting examples of vitamins include, but are not limited to, vitamin A, vitamin B₁, vitamin B₂, vitamin B₃, vitamin B₅, vitamin B₆, vitamin B₇, vitamin B₉, vitamin B₁₂, vitamin C, vitamin E, calcium, chromium, manganese, iron, or zinc.

Non-limiting examples of nutritional supplements include, but are not limited to, antioxidants, amino acids, green tea extract, creatine, alpha-lipoic acid, taurine, acai berry extract, pomegranate extract, lutein, guarana, choline, L-carnitine, coenzyme Q10, omega-3 fatty acids, pepsin, trypsin, carotenes, flavonoids, or polyphenols.

In some embodiments, the sports beverage may further comprise food-grade acids to adjust the pH of the beverage, including but not limited to citric acid, ascorbic acid, acetic acid, formic acid, butyric acid, fumaric acid, glycolic acid, lactic acid, malic acid, phosphoric acid, oxalic acid, succinic acid, and tartaric acid. In other embodiments, one or more food-grade acids may be used in combination.

In still other embodiments, the sports beverage may further comprise additives that serve as preservatives, including but not limited to sodium benzoate, potassium benzoate, sodium sorbate, potassium sorbate, ascorbic acid, citric acid, calcium propionate, sodium erythorbate, sodium nitrite, calcium sorbate, butylated hydroxyanisole, butylated hydroxytoluene, ethylenediaminetetraacetic acid (EDTA), tocopherols, straight chain polyphosphates, or combinations thereof.

In still other embodiments, the sports beverage may further comprise additives such as caffeine, flavor potentiators, micronutrients, plant extracts, phytochemicals, buffering salts, thickening agents, medicaments, or combinations thereof.

Also embodied herein are other performance enhancing foodstuffs, including a sports gel, a sports bar, a sports supplement, or other sports nutrient food. The sports gel, sports bar, sports supplement, or other sports nutrient foods may comprise a carbohydrate source in an amount ranging from about 1.0 wt % to about 30.0 wt %, an acetate source, and one or more electrolytes, including sodium in amounts and from sources comparable to those identified hereinabove.

The following examples are included to demonstrate preferred embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth in the following examples is to be interpreted as illustrative and not in a limiting sense.

### EXAMPLES

### Example 1

### Sports Beverage Compositions

Test Beverage A. Test Beverage A contained the following ingredients: water, citric acid, acetic acid (0.54 g acetate per 16 fl. Oz.), natural flavors, sodium chloride, sodium citrate, potassium citrate, potassium chloride, potassium phosphate, sucrose (6 wt %), niacinamide (vitamin B₃), pyridoxine hydrochloride (vitamin B₆), and cyanocobalamin (vitamin B₁₂). Test Beverage A has a total sodium content of 225 mg/L.

Test Beverage B. Test Beverage B contained the following ingredients: water, citric acid, triacetin (0.54 g acetate per 16 fl. Oz.), natural flavors, sodium chloride, sodium citrate, potassium citrate, potassium chloride, potassium phosphate, sucrose (6 wt %), niacinamide (vitamin B₃), pyridoxine hydrochloride (vitamin B₆), and cyanocobalamin (vitamin B₁₂). Test Beverage B has a total sodium content of 225 mg/L.

Control Beverage. Powerade® (The Coca-Cola Company, Atlanta, Ga.), which contained the following ingredients: water, citric acid, natural flavors, sodium chloride, sodium citrate, potassium citrate, potassium chloride, potassium phosphate, sucrose (6 wt %), niacinamide (vitamin B₃), pyridoxine hydrochloride (vitamin B₆), and cyanocobalamin (vitamin B₁₂).

### Comparative Testing of Work Capacity During Exercise in Trained Male Athletes

Trained male subjects 18-30 years of age who participated in regular cycling (defined as ≧60 minutes of cycling 4-5 days/week or 100 miles per week) were used in this example.

Endurance cycle test. Subjects performed a 60 minutes endurance cycling protocol using a cycle ergometer (Lode Corival V3; Groningen, Netherlands). The 60 minutes endurance protocol was divided into four 15-minute continuous segments. The first 45 minutes of this session (endurance segments 1-3) were submaximal and performed at 65% of the workload associated with each subject's VO_{2MAX}. The final 15 minutes (endurance segment 4) was a simulated “time trial” where subjects were instructed to generate as much work as possible, simulating the end of a race. Subjects were instructed to manually increase cycle resistance during this final segment as much as possible but still complete the entire protocol. Subjects consumed a serving of their assigned test beverage equivalent to 3 mL/kg body weight during each of the first three segments. Subjects were instructed to consume each serving at their own pace with the caveat that each serving must have been consumed prior to the beginning of the gas exchange measurements that were obtained during the last five minutes of each segment. Prior to the beginning of the endurance test and during the final five minutes of each segment, a blood sample was obtained, heart rate, blood pressure, electrocardiogram (ECG) for safety, and Rating of Percent Exertion (RPE) were assessed. Additionally, respiratory gases were measured on the metabolic cart during the final five minutes of segments 1-3 and continuously throughout endurance segment 4 until the completion of exercise.

Blood sampling and analysis. Blood samples were obtained at several time points with the use of a venous indwelling catheter inserted into an antecubital vein and held patent with periodic flushes of isotonic saline. A sample was obtained prior to consumption of breakfast and study beverage, following the post-breakfast rest period, prior to the beginning of the endurance cycle test, and at the end of each of the four endurance cycle test segments. Each sample was analyzed for glucose, lactate, free fatty acids, electrolytes (sodium, potassium, and chloride), bicarbonate, osmolality, and pH. Plasma glucose was determined by photometry using an Olympus analyzer (Beckman Coulter, Brea, Calif.) and a Roche hexokinase reagent. Plasma lactate was determined by photometry using an Olympus analyzer and a Roche reagent. Serum free fatty acids were determined by photometry using a Daytona analyzer (Randox Laboratories, Kearneysville, W. Va.) and a Wako reagent. Serum electrolytes were determined with the use of an ion-selective electrode (Abbott Point of Care, Princeton, N.J.). Serum bicarbonate was determined by photometry using an Olympus analyzer and a Roche CO₂-L reagent. Serum osmolality was measured with an Advanced Instruments osmometer (Norwood, Mass.) using freezing point determination. Whole blood pH was determined by an i-STAT® System (Abbott Point of Care, Princeton, N.J.).

Urine sampling and analysis. All urine produced was collected starting prior to initial beverage consumption. Urine samples were also collected following the completion of the endurance cycle test. Any additional urine output between collection time points was pooled and added to the subsequent time point. Fluid retention was calculated based on change in body weight, fluid consumed and total urine produced.

Hemodynamic measurements. Blood pressure and heart rate were obtained at several time points. Assessments occurred prior to consumption of breakfast and study beverage, following the post-breakfast rest period, prior to the beginning of the endurance cycle test, and at the end of each of the four endurance cycle test segments. Blood pressure was determined with the use of a manual sphygmomanometer and heart rate was determined with the use of a manual pulse assessment.

Questionnaires. A palatability questionnaire was administered twice: after consuming the initial beverage serving at breakfast and at the end of the endurance test. A gastrointestinal tolerability questionnaire was also completed at the end of the treatment visit to assess the presence and severity of selected GI symptoms, including gas/bloating, nausea, flatulence, diarrhea/loose stool, constipation, and cramping. Test Beverage A and Test Beverage B performed similarly to the control beverage.

### Results

TABLE 1
Blood markers of metabolism during the endurance cycle test.

| | | | | |
|---|---|---|---|---|
| Pre-Endurance | | | | |
| Test | Segment 1 | Segment 2 | Segment 3 | Segment 4 |

Glucose, mg/dL

| | | | | | |
|---|---|---|---|---|---|
| Control | 87.1 (5.5) | 72.6 (2.8) | 83.8 (4.6) | 94.6 (3.3) | 99.4 (4.2) |
| Test Beverage A | 85.4 (7.1) | 73.9 (4.8) | 88.6 (3.2) | 98.6 (4.7) | 105.0 (4.0) |
| Test Beverage B | 83.8 (3.1) | 69.6 (2.6) | 84.6 (3.2) | 97.7 (3.2) | 100.4 (3.6) |

Lactate, mg/dL

| | | | | | |
|---|---|---|---|---|---|
| Control | 40.1 (3.2) | 65.5 (7.5) | 73.0 (11.7) | 66.3 (9.9)¹ | 71.7 (6.3) |
| Test Beverage A | 38.3 (5.3) | 61.3 (7.9) | 67.9 (11.7) | 56.6 (8.6) | 70.4 (9.0) |
| Test Beverage B | 46.8 (6.7) | 57.6 (5.4) | 61.8 (8.2) | 58.9 (6.5) | 64.3 (4.4) |

As demonstrated by the results in Table 1, test subjects consuming either Test Beverage A or Test Beverage B had lower blood lactate levels compared to test subjects who consumed the Control Beverage.

TABLE 2
Fluid dynamics.

| | |
|---|---|
| Mean (SEM) | Median (interquartile limits) |

Total urine volume, mL

| | | | |
|---|---|---|---|
| Control | 757.3 (90.7) | 690.0 | (600.0, 1025) |
| Test Beverage A | 962.3 (118.4) | 950.0 | (800.0, 1400.0) |
| Test Beverage B | 980.5 (124.2) | 1075.0 | (575.0, 1350.0) |

Net fluid loss, mL

| | | | |
|---|---|---|---|
| Control | 549.0 (260.6) | 502.0 | (−348.0, 1320.0) |
| Test Beverage A | 43.6 (257.4) | 377.0 | (−488.0, 570.0) |
| Test Beverage B | 325.9 (244.1) | 307.0 | (−229.0, 627.0) |

As demonstrated by the results in Table 2, test subjects consuming either Test Beverage A or Test Beverage B had considerably less fluid loss compared to test subjects who consumed the Control Beverage.

TABLE 3
Respiratory and cardiovascular variables during the endurance cycle test.

| | | | | |
|---|---|---|---|---|
| Pre-Endurance | | | | |
| Test | Segment 1 | Segment 2 | Segment 3 | Segment 4 |

Minute ventilation, L/min

| | | | | | | |
|---|---|---|---|---|---|---|
| Control | 16.5 | (0.8) | 76.7 (4.5) | 80.4 (4.4) | 80.5 (4.8) | 89.3 (5.1) |
| Test Beverage A | 15.1 | (0.9) | 73.1 (3.5) | 79.6 (5.4) | 76.9 (3.9) | 87.9 (4.2) |
| Test Beverage B | 15.1 | (0.9) | 73.1 (3.5) | 76.7 (3.5) | 76.1 (3.4) | 84.7 (4.7) |

Rating of perceived exertion

| | | | | | | |
|---|---|---|---|---|---|---|
| Control | 6.1 | (0.1) | 12.2 (0.5) | 14.6 (0.5) | 16.4 (0.4) | 17.9 (0.3) |
| Test Beverage A | 6.6 | (0.3)¹ | 11.9 (0.7) | 15.0 (0.7) | 15.6 (0.5) | 17.5 (0.4) |
| Test Beverage B | 6.0 | (0.0) | 11.6 (0.6) | 14.1 (0.5) | 16.0 (0.6) | 18.2 (0.3) |

As demonstrated in Table 3, minute ventilation, or oxygen uptake measured as liters of air inspired per minute, was reduced for test subjects that consumed both Test Beverage A and Test Beverage B.

### Example 2

### Sports Beverage Compositions and Performance Analysis

In an additional, non-limiting example of one embodiment of the present invention, male cyclists (n=11; 24.3±0.6 years; VO_{2MAX} of 54.9±2.7 ml/kg/min) consumed isocaloric sports beverages containing citric acid (placebo), triacetin (TRI), or acetic acid (AA) in a double-blind, randomized, controlled crossover study. Subjects consumed 710 mL beverage and a standard breakfast beginning each test day AND performed two 30 second Wingate cycle tests separated by 4 minutes and consumed 7.5 ml/kg beverage while resting during a 60-min recovery period. Subjects then cycled for three 15-min consecutive segments at 65% VO_{2MAX}, followed by a 15 minute simulated “time trial.” Three ml/kg beverage was consumed during the endurance cycle test. Plasma glucose and lactate; serum free fatty acids, sodium, potassium, chloride, bicarbonate, osmolality; whole blood pH, urine osmolality and specific gravity were obtained at times throughout the day to assess markers of metabolism, and respiratory and cardiovascular variables were assessed during the time trial. Data were analyzed using repeated measures analysis of variance including subject and treatment as factors; Tukey's test was used for pairwise comparisons. Data are presented as means±SEM and p<0.05 was considered significant.

Results. There was no effect of beverage type on performance or blood markers of metabolism observed during the Wingate tests. During recovery, rating of perceived exertion was higher for TRI than AA (p=0.03), systolic blood pressure was lower for TRI than AA (p=0.03), and diastolic blood pressure was lower for TRI than AA (p=0.04) and tended to be lower for AA (p=0.07) than placebo. During the endurance test there were no significant effects of beverage type on blood markers of metabolism. Glucose decreased in all treatments after segment 1 and rebounded after segment 2. By the end of segment 4, glucose was higher than pre-endurance test levels in all treatments, and glucose tended to be higher with TRI compared to placebo (p=0.08). Lactate levels were generally lower during the endurance test in both acetate containing beverages versus placebo with a trend for TRI consumption to reduce lactate compared to placebo after segment 3 (p=0.06). There were no differences between treatments in respiratory and cardiovascular variables during the endurance test (p>0.05). Minute ventilation was reduced with AA after segment 3 (p=0.03), and triacetin (p=0.08) versus control. Acetic acid consumption tended to reduce total work versus placebo (p=0.06) during the time trial. There were no significant changes in urine specific gravity, urine osmolality levels, total urine volume, or net fluid loss throughout the day (p>0.05).

Conclusions. This study provides preliminary evidence to suggest that sports beverages containing acetate might have favorable effects on lactate and minute ventilation during submaximal endurance exercise in trained male athletes.

Not wishing to be bound by any theory, it is believed that the consumption of the sports beverage enhanced athletic performance by providing an available source of short chain fatty acids (i.e., acetate) as an alternative energy source to muscles during exercise. The enhanced athletic performance was demonstrated by reduced production of lactic acid, decreased quantity of fluid loss, decreased minute ventilation during exercise, and increased work output in a given time.

All of the compositions and methods disclosed and claimed herein can be made and executed without undue experimentation in light of the present disclosure. While the compositions and methods of this invention have been described in terms of the foregoing illustrative embodiments, it will be apparent to those of skill in the art that variations, changes, modifications, and alterations may be applied to the composition, methods, and in the steps or in the sequence of steps of the methods described herein, without departing from the true concept, spirit, and scope of the invention. More specifically, it will be apparent that certain agents, additives and ingredients that are similar according to their chemical, physiological and/or gustative properties may be substituted for the agents, additives and ingredients described herein while the same or similar results would be achieved. All such similar substitutes and modifications apparent to those skilled in the art are deemed to be within the spirit, scope, and concept of the invention as defined by the appended claims. The references cited herein, to the extent that they provide exemplary procedural or other details supplementary to those set forth herein, are specifically incorporated herein by reference.

1. A method of enhancing athletic performance comprising consuming a sports beverage before, during, or after endurance exercise, wherein the sports beverage comprises an aqueous solution of:
a carbohydrate source in an amount ranging from about 1.0 wt % to about 10.0 wt % of the sports beverage;
an acetate source in an amount ranging from about 5.0 mM/L to about 40.0 mM/L; and
one or more electrolytes in an amount ranging from about 30 mM/L to about 180 mM/L.

2. The method of claim 1, wherein said carbohydrate source is selected from the group consisting of high fructose corn syrup, sucrose, fructose, maltodextrin, glucose, mono-saccharides, dextrose, maltose, dextrin, xylose, ribose, mannose, galactose, lactose, invert sugar, tagatose, sugar alcohols such as glycerol, sorbitol, xylitol, mannitol, galactitol, maltitol, lactitol, erythritol, hydrogenated starch hydrolase, polyglycitol, stevia, fruit juice and combinations thereof.

3. The method of claim 1, wherein said acetate source is selected from the group consisting of acetic acid, acetate, vinegar, acetate anhydride, calcium acetate, potassium acetate, sodium acetate, triacetin, propionic acid, isobutyric acid, butyric acid, isovaleric acid, valeric acid, caproic acid, lactic acid, lactate and combinations thereof.

4. The method of claim 1, wherein said electrolytes are selected from the group consisting of sodium, potassium, chloride, calcium, magnesium, bicarbonate, phosphate, sulfate, and combinations thereof.

5. The method of claim 1, wherein said carbohydrate source is present in an amount ranging from about 2.0 wt % to about 8.0 wt % of said sports beverage.

6. The method of claim 1, wherein said carbohydrate source is present in an amount ranging from about 4.0 wt % to about 8.0 wt % of the sports beverage.

7. The method of claim 1, wherein said carbohydrate source is high fructose corn syrup.

8. The method of claim 1, wherein said acetate source is present in an amount ranging from about 3.0 mM/L to about 28.0 mM/L.

9. The method of claim 1, wherein said sports beverage further comprises one or more coloring additives, one or more flavor additives, one or more artificial or non-caloric sweeteners, one or more vitamins, one or more nutritional supplements, and combinations thereof.

10. The method of claim 1, wherein said enhancing athletic performance is characterized by reduced perceived exhaustion, reduced lactic acid production, reduced muscle soreness, reduced muscle damage, reduced net fluid loss, or combinations thereof.

11. A sports beverage comprising an aqueous solution of:
a carbohydrate source in an amount ranging from about 1.0 wt % to about 10.0 wt % of the sports beverage;
an acetate source in an amount ranging from about 5.0 mM/L to about 40.0 mM/L; and
one or more electrolytes, including sodium in amount ranging from about 30 mM/L to about 180 mM/L.

12. The sports beverage of claim 11, wherein said carbohydrate source is selected from the group consisting of high fructose corn syrup, sucrose, fructose, maltodextrin, glucose, fruit juice, and combinations thereof.

13. The sports beverage of claim 11, wherein said acetate source is selected from the group consisting of acetic acid, acetate, vinegar, acetate anhydride, calcium acetate, potassium acetate, sodium acetate, triacetin, and combinations thereof.

14. The sports beverage of claim 11, wherein said electrolytes are selected from the group consisting of sodium, potassium, chloride, calcium, magnesium, bicarbonate, phosphate, sulfate, and combinations thereof.

15. The sports beverage of claim 11, wherein said carbohydrate source is present in an amount ranging from about 2.0 wt % to about 8.0 wt % of the sports beverage.

16. The sports beverage of claim 11, wherein said carbohydrate source is high fructose corn syrup.

17. The sports beverage of claim 11, wherein said acetate source is present in an amount ranging from about 3.0 mM/L to about 6.0 mM/L.

18. The sports beverage of claim 11, further comprising one or more coloring additives, one or more flavor additives, one or more artificial or non-caloric sweeteners, one or more vitamins, one or more nutritional supplements, and combinations thereof.

19. A method for enhancing performance comprising orally administering the sports beverage of claim 11 to an endurance athlete before, during, or after said endurance athlete engages in endurance exercise, wherein the enhanced performance is characterized by reduced perceived physical exhaustion, reduced muscle soreness, reduced muscle damage, reduced net fluid loss, reduced production of lactic acid, or combinations thereof.

20. A beverage comprising an aqueous solution of:
a carbohydrate source in an amount ranging from about 1.0 wt % to about 10.0 wt % of the sports beverage, wherein said carbohydrate source is selected from the group consisting of high fructose corn syrup, sucrose, fructose, maltodextrin, glucose, mono-saccharides, dextrose, maltose, dextrin, xylose, ribose, mannose, galactose, lactose, invert sugar, tagatose, sugar alcohols such as glycerol, sorbitol, xylitol, mannitol, galactitol, maltitol, lactitol, erythritol, hydrogenated starch hydrolase, polyglycitol, stevia, fruit juice and combinations thereof;
an acetate source in an amount ranging from about 5.0 mM/L to about 40.0 mM/L, wherein said acetate source is selected from the group consisting of acetic acid, acetate, vinegar, acetate anhydride, calcium acetate, potassium acetate, sodium acetate, triacetin, propionic acid, isobutyric acid, butyric acid, isovaleric acid, valeric acid, caproic acid, lactic acid, lactate and combinations thereof; and
one or more electrolytes in an amount ranging from about 30 mM/L to about 180 mM/L.
